(19) United States
(12) Patent Application Publication
(10) Pub. No.: US 2021/0158923 A1
(43) Pub. Date: May 27, 2021

(54) DISTRIBUTED USER MONITORING SYSTEM

(71) Applicant: CHANGING HEALTH LIMITED, Newcastle Upon Tyne (GB)

(72) Inventors: Michael TRENELL, Tyne and Wear (GB); Michael CATT, Northamptonshire (GB)

(21) Appl. No.: 16/610,720

(22) PCT Filed: May 4, 2018
(86) PCT No.: PCT/GB2018/051216

§ 371 Date: Nov. 4, 2019

(30) Foreign Application Priority Data
May 4, 2017 (GB) 1707119.2

(51) Int. Cl. G16H 20/00; G16H 50/20; G16H 50/30; G16H 10/60

(52) U.S. Cl. CPC G16H 20/00; G16H 10/60; G16H 50/30; G16H 50/20

(57) ABSTRACT

The present invention relates to a system and devices for monitoring medical conditions of patients. More particularly, the present invention relates to the monitoring of the behaviours and relevant diagnostics data relating to patients with or at risk of type 2 diabetes, and its associated cardiovascular disease and/or weight management, including using in-vitro diagnostics (IVD) devices. According to a first aspect, there is provided a method of monitoring one or more users comprising the steps of: receiving user variable data for each of the one or more users; receiving data from one or more user devices for each of the one or more users; predicting effectiveness data for each of the one or more users based on the user variable data for each of the one or more users; comparing received data with predicted effectiveness data for each of the one or more users; and determining an effectiveness value for each of the one or more users based on the comparison of the received data with the predicted effectiveness data.

### FIELD

The present invention relates to a system and devices for monitoring users. More particularly, the present invention relates to the monitoring of the behaviours and relevant diagnostics data relating to users, for example users with or at risk of type 2 diabetes and its associated cardiovascular disease and/or weight management, including using in-vitro diagnostics (IVD) devices.

### BACKGROUND

At present, it is thought that approximately 415 million people worldwide have diabetes and that most of these people have type 2 diabetes. In high-income countries, it is thought that up to 91% of adults with the disease have type 2 diabetes.

Higher levels of blood glucose also damage blood vessels and therefore patients with diabetes have a higher chance of developing cardiovascular disease. Another problem associated with diabetes is obesity. Due to the number of associated problems surrounding diabetes, around 12% of global health expenditure is dedicated to diabetes treatment and related complications. Many countries spend between 5% and 20% of their total health expenditure on diabetes.

It is the opinion of many key stakeholders that diabetes is one of the largest global health emergencies of the 21^{st} century. In addition to the approximately 415 million adults who currently have diabetes globally, there are 318 million adults who are at high risk of developing diabetes in the future. The incidence of type 2 diabetes continues to increase globally driven by increasing obesity rates.

Typically, individuals diagnosed with type 2 diabetes are managed by their respective health systems using well established clinical pathways. These clinical pathways initially attempt to manage blood glucose levels through lifestyle changes, including weight loss and increased physical activity, with progressive introduction of pharmacotherapy until sufficient control is established or maintained. In some cases, surgery may be required.

An increasing range of pharmacological therapies are becoming available for type 2 diabetes management. Diabetes control is measured by looking at the amount of sugar absorbed into the blood. Glycosylated haemoglobin (HbA1c) is the core biomarker identified for assessing blood glucose control and is typically measured in individuals at regular intervals. HbA1c forms when glucose reacts with haemoglobin in the red blood cells and represents the aggregate exposure to glucose in the 8 to 12 week lifetime of a typical red blood cell.

Typically, clinical guidelines do not recommend the use of home blood glucose monitoring for individuals with type 2 diabetes except under specific circumstances—instead periodic assessments for other disease indicators and consequences are made by medical professionals, including: body mass index measurement, blood pressure measurement, HbA1c measurement, cholesterol measurement, smoking status, foot examination, albumin-creatinine ratio and serum creatinine measurement; but the comprehensiveness and periodicity of these assessments can be very variable between individuals as typically assessments are performed only on an annual basis.

Drug-based therapy is generally initiated using drugs like Metformin and intensified by combining Metformin with other drugs, with alternatives for individuals unsuited to Metformin. Drug-based therapy is generally acknowledged to delay transition to, or progression of type 2 diabetes. Currently, the American Diabetes Association (ADA), the European Association for the Study of Diabetes (EASD) and the UK Prospective Diabetes Study (UKPDS) have all published various treatment plans and guidelines for managing and treating diabetes, including the use of combining Metformin and another drug.

Current diabetes management can be characterised by the inadequate delivery of lifestyle management advice and implementation of any advice by patients, a lack of a formalised path in clinical guidelines towards “diabetes reversal” where drug doses can be reduced or withdrawn, infrequent monitoring of blood glucose control for most patients, inadequate monitoring of both lifestyle and medication conformance, effectiveness, side-effects and cost effectiveness, and the variable delivery of periodic assessments required to detect disease progression or the emergence of complications.

### SUMMARY OF INVENTION

Aspects and/or embodiments seek to provide a method and/or system for the monitoring of users based on data collected by user devices compared to the predicted data expected for that user. Further aspects and/or embodiments related to the use of in-vitro diagnostics in this regards, and/or for the improved assessment and management of people having type 2 diabetes by regimes incorporating both lifestyle modification and pharmacotherapy.

According to a first aspect, there is provided a method of monitoring one or more users comprising the steps of: receiving user variable data for each of the one or more users; receiving data from one or more user devices for each of the one or more users; predicting effectiveness data for each of the one or more users based on the user variable data for each of the one or more users; comparing received data with predicted effectiveness data for each of the one or more users; and determining an effectiveness value for each of the one or more users based on the comparison of the received data with the predicted effectiveness data.

Monitoring effectiveness, for example of a patient's (or user's) treatment and/or behaviour and/or regime such as exercise regime, can be accomplished by collecting user data in real-time or near real-time and comparing this to one or more predictions in relation to the user based on characteristics and/or prior information and/or other variables for that or each user. Rather than having to wait for or go through the costly process of scheduled in-person appointments, for example to visit a medical centre and/or personal trainer and/or coach, the user's data and/or variables stored for that user—for example in a digital medical record (stored on a database)—can be updated instantly by measurements or data received through the user's device(s) to allow remote access and/or automated processing. This collected data can be used to provide an instant determination in relation to monitoring the user, for example by predicting or having predicted (and/or updating said predictions of) what data is expected for a user at any given time and/or over any given time period and comparing this prediction to the actual data collected from one or more user devices, for example to assess or determine of the effectiveness of the patient's current treatment plan and/or behaviour and/or regime such as an exercise regime, for which information/feedback/intervention can then be tailored and/or adapted accordingly perhaps even substantially immediately. Example variables for a user are not intended to be limited to but can include any of: biometric data; biological data; medical data; physiological data and personal data.

Optionally, the one or more user devices include in-vitro devices, smartphones, tablet computers, personal computers, smart watches, smart glasses, in-ear computers, sensors integrated into clothing or apparel, or other similar devices to capture data.

IVD devices can commonly be used to perform tests on samples taken from the human body (for example, blood, urine or tissue). Other user devices can allow for a more holistic, or more complete data “picture” to be formed for a user.

Optionally, the method includes a step of determining a state for each of the one or more users wherein the state correlates to a need for treatment.

In determining a state which correlates to a need for treatment, users may be managed to prevent or lower the risk of type 2 diabetes without the user needing to delay for consultation by a clinical care team. Further, by determining a particular level or stage of risk of a user in being diagnosed with type 2 diabetes, prevention regime(s) can be provided accordingly.

Optionally, the treatment comprises a lifestyle regime and/or a drug(s) regime.

Both regimes can be used to prevent, manage or reverse type 2 diabetes. Furthermore, the effectiveness value established from the comparison step may determine a combination of or a modification of a lifestyle regime and/or a drug(s) regime. Optionally, the regimes can manage or treat type 1 and/or type 2 diabetes. Optionally other regimes might include for example an exercise regime or a dietary regime.

Optionally, the data from one or more user devices is input manually by the user and/or collected as sensor data from the one or more user devices.

Enabling the user to input data can enhance the data collected rather than only using the (sometimes limited) automatic data collection features offered by IVD devices, smartphones, or the like. For example, manual data can be collected in relation to diet or meditation or state of mind.

Optionally, the predicted effectiveness data corresponds to responses to a drug(s) regime and/or a goal-orientated response to a lifestyle regime. Optionally, the predicted effectiveness data corresponds to other regimes.

The predicted effectiveness data can be used as a target for a user to achieve or a threshold value of interested, both of which may be set by a clinician/doctor/medical supervisor with the user. Any new user data may therefore be compared to this predicted effectiveness data.

Optionally, the further step of determining negative responses to the treatment is performed.

This negative response can allow a straightforward indication of when a treatment and/or lifestyle regime is not effective, for example.

Optionally, the method includes a step of categorising the one or more users into one or more user categories based on the responses to the drug(s) regime and/or the goal orientated response to a lifestyle regime. Optionally, one or more commonalties is determined for each of the one or more users in the one or more categories and/or in order to categorise users into categories.

Segmentation of users may be done using the application of artificial intelligence and/or machine learning techniques to an individual's data to determine a level of anticipated engagement. For example, training data in relation to users and/or regimes and/or effectiveness can be used to develop neural networks such as convolutional neural networks that can classify users into categories based on the user data. Further, by categorising users in accordance to user response, and determining one or more commonalities between users within user categories, data and/or training data may be built up in order to better determine the goal-orientated lifestyle and/or drug regime for users who fall within said categories.

Optionally, the user data comprises any type of biomarker data.

Use of a variety of biomarker data that is collected either by a medical professional or by a user can provide a richer data set for the user from which to allow assessment of the user treatment and/or lifestyle regime.

Optionally, the treatment of first aspect of the invention comprises the use of any of SGLT2 inhibitors, DPP-4 inhibitors and GLP-1 receptor agonists.

All three of these drug classes can be used to manage or treat diabetes.

According to a second aspect, there is provided a method of monitoring one or more users in respect to conformance with a treatment, comprising the steps of: receiving user variable data for each of the one or more users; receiving data from one or more user devices for each of the one or more users; predicting effectiveness data for each of the one or more users based on the user variable data for each of the one or more users; comparing received data with predicted effectiveness data; determining conformance with the treatment for each of the one or more users based on the comparison of the received data with the predicted effectiveness data based on the comparison.

By automatically assessing the user conformance with a treatment in real-time using data received by the users relative to a set of predicted and/or predetermined data, the system and/or a user can substantially instantly and/or at intervals determine whether the treatment and/or lifestyle regime is appropriate for the user.

Optionally, the treatment plan comprises a lifestyle regime and/or a drug(s) regime. Optionally, the received data is compared to any one of predicted effectiveness data and predetermined conformance data, wherein the predicted effectiveness data and predetermined conformance data corresponds to a response to a drug(s) regime and/or a goal-orientated response to a lifestyle regime.

Optionally, the step of determining conformance of the treatment based on the comparison further determines a modification of the lifestyle regime and/or the drug(s) regime and the treatment further comprises the use of any of SGLT2 inhibitors, DPP-4 inhibitors, GLP-1 receptor agonists.

The system therefore can promote a cost-effective method of treating diabetes by determining the optimal combination of a lifestyle regime and a pharmacotherapy regime by continuously gathering user data and having the data analysed with respect to targets and goals to determine the effectiveness of treatments. The system can provide a tailored or personal approach to each individual user.

According to a third aspect, there is provided a method of determining a treatment for one or more users comprising the steps of: receiving user variable data for each of the one or more users; receiving data from one or more user devices for each of the one or more users; predicting effectiveness data for each of the one or more users based on the user variable data for each of the one or more users; comparing the received data with predicted effectiveness data; determining an effectiveness value for each of the one or more users based on the comparison of the received data with the predicted effectiveness data based on the comparison; and determining the treatment for the one or more users in dependence upon the effectiveness value.

The system can automatically suggest/select a treatment for a user using an effectiveness value derived from real-time data provided by the users. In this way, a suggested treatment plan can be reviewed by a user's clinician/doctor/medical advisor without having to wait to meet the user or obtain measurements from the user directly. This can provide a more streamlined and cost-effective consultation process when needing to amend or change a user's treatment type.

Optionally, the treatment comprises a lifestyle regime and/or a drug(s) regime, the predicted effectiveness data corresponds to a response to a drug(s) regime and/or a goal-orientated response to a lifestyle regime.

Optionally, the predicted effectiveness data further corresponds to the one or more users' conformance with the treatment.

In order to understand whether a diabetes drug has been effective it is important to know in the first instance if they have been taken. Once conformance and/or the physiological level of the drug is known, efficacy can be determined by understanding the effect on diabetes control, but only after controlling for lifestyle regimes such as diet, physiological activity and/or sleep.

Optionally, further comprising a step of categorising the one or more users into one or more user categories based on the responses to the drug(s) regime and/or the goal orientated response to a lifestyle regime and/or the one or more users conformance with the treatment.

Optionally, one or more commonalities is determined for each of the one or more users in the one or more users categories, wherein the one or more commonalities determines a modification of the lifestyle regime and/or the drug(s) regime and the treatment further comprises the use of any of SGLT2 inhibitors, DPP-4 inhibitors, GLP-1 receptor agonists.

By categorising users in accordance to user response, and determining one or more commonalities between user categories, data may be built up in order to better determine the goal-orientated lifestyle and/or drug regime for users who fall within said categories.

Optionally, the method of treatment comprises treatment and/or management of users diagnosed with or at risk of type 2 diabetes.

According to a fourth aspect, there is provided a method of monitoring the effectiveness of a treatment in one or more patients comprising the steps of receiving data from one or more patient devices, comparing received data with predetermined effectiveness data and determining an effectiveness value based on the comparison.

Monitoring the effectiveness of a patient's treatment can be accomplished by collecting patient data in real-time or near real-time. Rather than having to wait for or go through the costly process of scheduled appointments to visit a medical centre, the patient's digital medical record (stored on a database) can be updated instantly by measurements or data received through the user's device(s). This data can then be used to provide an instant determination of the effectiveness of the patient's current treatment plan, which can then be immediately tailored accordingly.

Optionally, the one or more patient devices include in-vitro devices, smartphones, tablet computers, personal computers or other similar devices to capture data.

IVD devices can commonly be used to perform tests on samples taken from the human body (for example, blood, urine or tissue).

Optionally, the patient treatment comprises a lifestyle regime and/or a drug(s) regime.

Both regimes can be used to prevent, manage or reverse type 2 diabetes. Furthermore, the effectiveness value established from the comparison step may determine a combination of or a modification of a lifestyle regime and/or a drug(s) regime. Optionally, the regimes can manage or treat type 1 and/or type 2 diabetes.

Optionally, the data from one or more patient devices is input manually by the patient and/or collected as sensor data from the patient device.

Enabling the patient to input data can enhance the data collected rather than only using the sometimes limited automatic data collection features offered by IVD devices, smartphones, or the like.

Optionally, the predetermined effectiveness data corresponds to responses to a drug(s) regime and/or a goal-orientated responses to a lifestyle regime.

The predetermined effectiveness data can be used as a target for a patient to achieve or a threshold value of interested, both of which may be set by a clinician/doctor/medical supervisor with the user. Any new user data may therefore be compared to this predetermined effectiveness data.

Optionally, the further step of determining negative responses to the treatment is performed.

This negative response can allow a straightforward indication of when a treatment and/or lifestyle regime is not effective, for example.

Optionally, the user data comprises any type of biomarker data.

Use of a variety of biomarker data that is collected either by a medical professional or by a user can provide a richer data set for the patient from which to allow assessment of the patient treatment and/or lifestyle regime.

Optionally, the treatment of first aspect of the invention comprises the use of any of SGLT2 inhibitors, DPP-4 inhibitors and GLP-1 receptor agonists.

All three of these drug classes can be used to manage or treat patient with diabetes.

According to a fifth aspect, there is provided a method of monitoring patient conformance with a treatment, comprising the steps of receiving data from one or more patients, comparing the received data with predetermined data and determining conformance of the treatment based on the comparison.

By automatically assessing the patient conformance with a treatment in real-time using data received by the patients relative to a set of predetermined data, the system and/or a user (such as a medical professional) can substantially instantly and/or at intervals determine whether the treatment and/or lifestyle regime is appropriate for the patient.

Optionally, the treatment plan comprises a lifestyle regime and/or a drug(s) regime, the predetermined data corresponds to a response to a drug(s) regime and/or a goal-orientated response to a lifestyle regime and the treatment further comprises the use of any of SGLT2 inhibitors, DPP-4 inhibitors, GLP-1 receptor agonists.

According to a sixth aspect, there is provided a method of selecting a treatment for a patient comprising the steps of receiving data from one or more patients, comparing the received data with predetermined effectiveness data, outputting an effectiveness value based on the comparison and selecting a treatment in dependence upon the effectiveness value.

The system can automatically suggest/select a treatment for a patient using an effectiveness value derived from real-time data provided by the patients. In this way, a suggested treatment plan can be reviewed by a patient's clinician/doctor/medical advisor without having to wait to meet the patient or obtain measurements from the patient directly. This can provide a more streamlined and cost-effective consultation process when needing to amend or change a patient's treatment type.

Optionally, the treatment comprises a lifestyle regime and/or a drug(s) regime, the predetermined effectiveness data corresponds to a response to a drug(s) regime and/or a goal-orientated response to a lifestyle regime and the treatment further comprises the use of any of SGLT2 inhibitors, DPP-4 inhibitors, GLP-1 receptor agonists.

Optionally, the method of treatment comprises treatment and/or management of type 2 diabetes.

According to a seventh aspect, there is provided an apparatus operable to perform the method of any preceding feature.

### BRIEF DESCRIPTION OF DRAWINGS

Embodiments will now be described, by way of example only and with reference to the accompanying drawings having like-reference numerals, in which:

FIG. 1 illustrates an embodiment of the system where users can interact with the system via personal devices and the system can interact with one or more medical records; and

FIG. 2 illustrates the use of a tailored care pathway based on an individuals' behavioural and physiological response.

### SPECIFIC DESCRIPTION

While lifestyle modification is advocated for people at risk of developing diabetes (for example, people with elevated glucose levels, non-alcoholic fatty liver disease or people with evidence of impaired energy metabolism), pharmacotherapy is typically reserved for people diagnosed with diabetes per accepted clinical criteria.

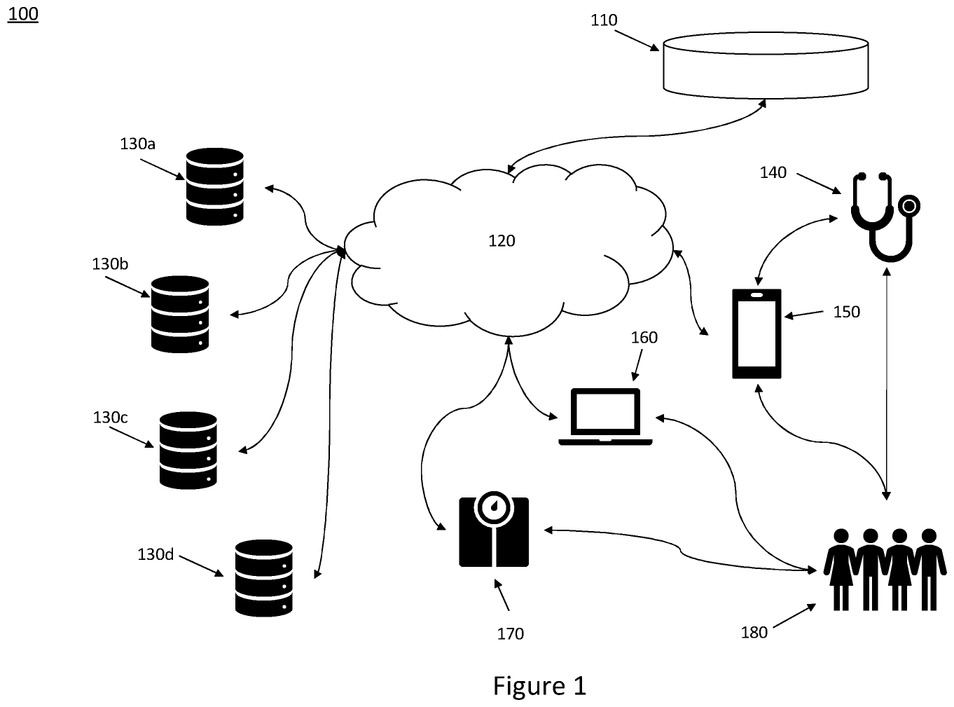

Referring to FIG. 1, an example embodiment will now be described.

The system 100 of this embodiment operates over a distributed network 120, such as the internet, to allow user devices 150, 160, 170 to communicate with a server 110 and the server to communicate with one or more databases holding medical records 130a, 130b, 130c, 130d.

In other embodiments, other suitable communications networks instead or as well as the internet can be used to allow the system 100, user devices 150, 160, 170, server 110 and databases holding medical records 130a, 130b, 130c, 130d to communicate. Such suitable communications networks might include mobile ‘phone data networks, mesh networks or wireless local area networks. In some embodiments, the server 110 may not be a single physical server but may instead be a virtual device or a cloud-implemented distributed data service. User devices 150, 160, 170 can include mobile phones 150, personal computers 160 and connected devices 170. In some embodiments, user devices can also include devices 140 that do not connect directly to the distributed network 120 but can connect via a user device 150, 160, 170 using a wired and/or wireless connection, and/or by transferring data using physical means such as using a memory card and/or device, and/or QR code and/or other symbol capable of machine reading and/or manual input by a user 180.

In some embodiments, directly connected devices 170 and indirectly connected devices 140 can include in-vitro diagnostics devices that users 180 can use to measure and record various medical characteristics, including for example in-vitro diagnostics devices to perform any of urine tests, blood-glucose tests, blood pressure test, and other tests.

In this embodiment, the system 100 provides a tool for users 180 and medical professionals to assess and/or manage people with type 2 diabetes including by use of regimes incorporating both lifestyle modification and pharmacotherapy. In comparison to type 1 diabetes, there are several options for treating type 2 diabetes. More particularly, type 2 diabetes is managed over longer time periods in order to properly determine the effectiveness of a treatment plan, whereas a patient with type 1 diabetes needs to be monitored almost hourly.

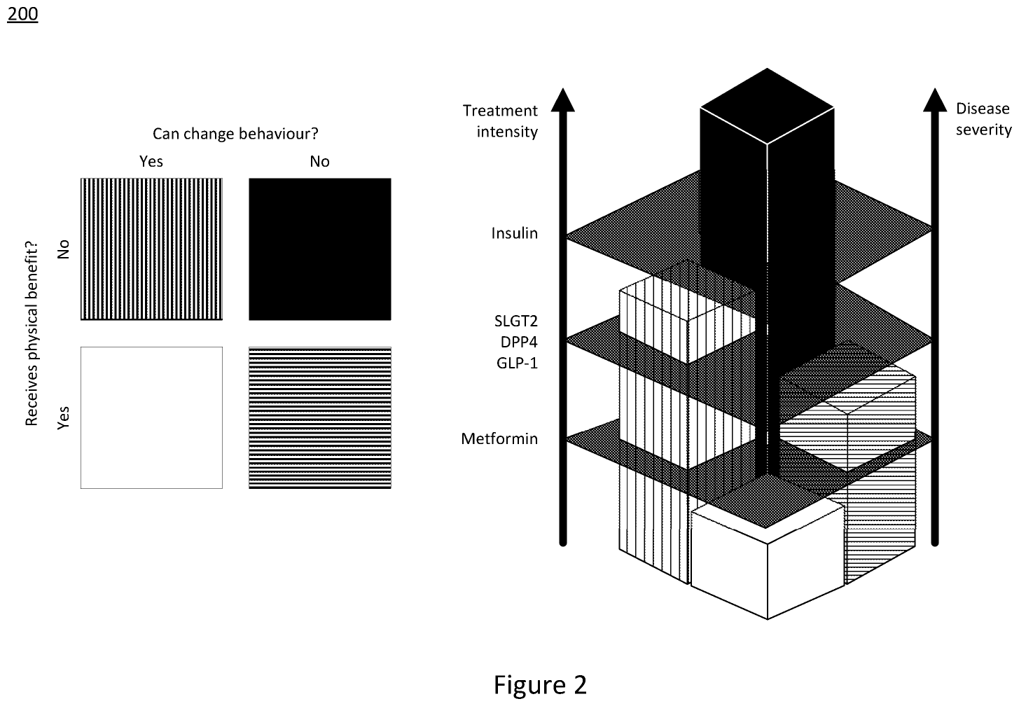

Both regimes, lifestyle regimes and/or drug regimes, can be used to prevent, manage or reverse type 2 diabetes. For example, the use of blood glucose monitoring can help provide immediate information on current diabetes control. This data can be utilised to establish whether current treatment approaches are adequate or not. This establishment is provided as output from the system. The monitoring of behaviour, such as diet, physical activity and/or sleep, can provide feedback on adequate behaviour change, which may further act as input into the system. Linked with glucose monitoring, the system may be capable of notifying the user whether their lifestyle change(s) are adequate or not—creating a behavioural feedback loop. If lifestyle changes are seen to be insufficient the user may take drugs as part of their regime for additional support. Drug use can tailor advice and give information about the effectiveness regarding the drug. Drug adherence can be monitored by using an IVD detecting the compound levels or by time annotated photographs, i.e. images with time and date stamps, which shows the user's consumption of the drug. Through the combination of regimes, this system gives a dynamic and real time framework for creation of a personalised treatment pathway for the prevention or management of type 2 diabetes supported by IVD and behavioural sensors/data. As a representation, FIG. 2 serves to illustrates the use of a tailored care pathway based on an individuals' behavioural and physiological response.

The system, in some embodiments, can function to assist medical professionals to devise new approaches to the selection, use and therapy monitoring of new generations of diabetes drugs including but not limited to, for example, SGLT2 inhibitors, DPP-4 inhibitors and GLP-1 receptor agonists. Further, in these and other embodiments the system can assist with the combined management of diabetes in patients via lifestyle modification (including for example diet, physical activity, and sleep) and pharmacotherapy. To understand whether a diabetes drug has been effective it is important to know in the first instance if they have been taken. The system will establish taking of drugs it its simplest format, by images with time stamps. A more advanced configuration may be detection of drug dose within a physiological sample such as blood, urine or saliva using an IVD. The IVD will link back to the system via a connection with a mobile device or the internet. The IVD may establish the circulatory physiological levels of the drug. Once conformance and/or the physiological level of the drug is known, efficacy can be determined by understanding the effect on diabetes control, but only after controlling for lifestyle regimes such as diet, physiological activity and/or sleep.

Users 180 have at least one device 140, 150, 160, 170 that can interact with the system 100 and the server 110 in the embodiments. In the exemplary embodiment, the users 180 each have a mobile ‘phone 150 that connects wirelessly to one or more in-vitro diagnostic devices (which can include a blood glucose testing device and/or a urine testing device) and a personal computer 160 and one or more connected personal biosensors (which can include a set of scales, a blood pressure monitor, a heart rate monitor). In other embodiments, users 180 may have a variety of combinations of these user devices 140, 150, 160, 170.

In some embodiments, some or all of the users 180 can have digital medical record data stored by their medical professionals in one or more databases 130a, 130b, 130c, 130d. In some embodiments, the server 110 is in communication over the distributed network 120 with one of more of the digital medical record databases 130a, 130b, 130c, 130d to access some or all of the digital records for at least a portion of the some or all users 180 having digital medical record data. In some embodiments, accessing the digital records can include either or both of retrieving information from the databases 130a, 130b, 130c, 130d and/or entering or updating information into or in the databases 130a, 130b, 130c, 130d.

An example user interaction with the system 100 according to an embodiment will now be described.

In this example, each user 180 has digital medical records stored in several databases 130a, 130b, 130c, 130d. Each database is held by a different medical organisation, for example one database 130a may be held by the user's local doctor's surgery or general practitioner while other databases 130b, 130c, 130d may be held at a hospital, insurance company and on a central government health record. The server 110 may be in communication with one or more of these databases 130a, 130b, 130c, 130d to maintain a synchronised redacted or full medical record for one or more users 180 at the server. Further, information stored on the server 110 for the users 180 can be updated or inserted into the digital medical records in one of more of the databases 130a, 130b, 130c, 130d.

Upon assessment or testing by a medical professional, such as at their local doctor's surgery by a general practitioner, a user may be diagnosed as at risk or already having type 2 diabetes. One or more of their medical records 130a, 130b, 130c, 130d will be updated accordingly.

Following such a diagnosis, the patient may then be issued with, or acquire, one or more IVD tools 140. The IVD tools 140 may wirelessly connect (or the user can somehow transfer the data automatically or manually) to a device such as a ‘phone 150 or computer 160 of the user 180. The IVD tools may alternatively be connected also to the internet. The IVD tool 140 may be operated by the user 180 on a daily, weekly, periodic, episodic or random sampling regime. Using their ‘phone 150 or computer 160 the user 180 can automatically or manually allow the data from the IVD tools 140 to be transferred over the network 120 to the server 110.

The IVD tools 140 may comprise an IVD reader and a separate IVD test device (not shown). The user 180 may use the IVD test device which may be a single or multi-analyte device to record data relating to treatments using SGLT2 inhibitors, DPP-4 inhibitors or in response to a lifestyle regime.

Whether a user 180 is diagnosed with diabetes or identified as an at-risk diabetes patient, in some embodiments, using the data acquired by the user devices 140, 150, 160, 170, the system 100 determines the likely clinical effectiveness of lifestyle modification alone. Preferably, the effectiveness of lifestyle modification alone can be assessed by analysing the fasting glucose levels over time. Other factors such as psychological measures of preparedness, mood and outlook contributing to the likelihood of achieving concordance with a lifestyle modification regime together with other complicating physiological, existing multi-morbidity or complications, physical capability, social and environmental factors may also be recorded by the user 180.

For users 180 unresponsive to lifestyle modification alone, the system will conduct an early escalation to recommend a combined regime of lifestyle and pharmacotherapy. Examples of an unresponsive user could be either someone who is unable to achieve a behavioural change goal in physical activity, weight, or sleep over a period of time or someone who is able achieve the behavioural change goal in physical activity, weight, or sleep but through which had no effect on diabetes control. Additional segmentation of users may be done using the application of artificial intelligence to an individual's data to determine a level of anticipated engagement. In both cases, the unresponsive user would be directed to additional therapy with the support of drugs to achieve better diabetes control. If the drug is ineffective it may prompt the user to visit their care team to increase the dose or use other therapeutic options. If a user achieves significant behavioural change, i.e. loses significant weight loss, the system could support reduction or withdrawal of drugs. This is counter to the existing treatment pathway where in such cases the user visits a clinical care team every 6 months for example where they are informed how effective their lifestyle or drug therapy is in managing their diabetes for the previous 6 months.

On the other hand, users 180 that are identified to respond positively to lifestyle modification alone and with the likelihood of low concordance between a lifestyle regime and a pharmacotherapy regime, the system 100 automatically encourages the user to continue with the lifestyle modification alone. A positive responder to lifestyle modification will be determined by both the ability to achieve a behavioural change, regarding for example, physical activity, diet, sleep, and/or weight, and measures of diabetes control target HbA1c range and/or disease status targets such as the restoring normal fasting glucose and/or normal/improved glycaemic response to carbohydrate intake.

Additionally, the system 100 determines the likelihood of a user 180 to achieve diabetes reversal, establish a stable disease state or delay the progression of diabetes via lifestyle modification alone. The system can monitor the conformance a lifestyle regime by comparing patient data to a target HbA1c range and/or disease status targets (for example, the restoring normal fasting glucose and/or normal/improved glycaemic response to carbohydrate intake). These targets may be predetermined by a clinical care team with the patient.

Lifestyle regimes can be monitored and adjusted in real time through the user devices and by means of self-reporting, personal biosensor (for example, movement, diet, heart rate measurements, etc) and IVD measures (for example, episodic continuous glucose monitoring) allowing the patient and care team to observe the specific impact of behaviours on glucose control and other outcomes.

The system can allow for early detection and management of effectiveness of types of therapies, compliance issues, side effects and adverse effects. The breadth of data collected by the system allows for early detection and management of effectiveness of types of therapies, compliance issues, side effects and adverse effects. The breadth of data collected by the system may identify novel ways of identifying effectiveness, compliance, side effects or adverse events. For example, sleep disruption as a side effect or sleep improvement as a measure of efficacy. Changes in everyday physical activity patterns as a measure of efficacy or a side effects.

In some embodiments it is only in the case, where a user does not respond to lifestyle modifications alone, that the system indicates a treatment using a combination of lifestyle modifications and pharmacotherapy. These embodiments seek to avoid patients being prescribed medication until other alternatives have been used initially.

In some embodiments, the system provides real-time feedback to the users 180 by presenting them with the impact of their treatments and/or lifestyle regime. This can increase and/or sustain the concordance and clinical effectiveness of the treatment/lifestyle regime, can reduce the automatic primary escalation to the more expensive combination of lifestyle modification and pharmacotherapy regime (as well as a successive escalation to polypharmacotherapy), and the eventual secondary escalation to the introduction of insulin.

The system therefore can promote a cost-effective method of treating diabetes by determining the optimal combination of a lifestyle regime and a pharmacotherapy regime by continuously gathering user data and having the data analysed with respect to targets and goals to determine the effectiveness of treatments. The system of at least some embodiments can provide a tailored or personal approach to each individual user. For short term goal orientation, if a drug is effective it would provide feedback to the user as such helping reinforce taking the drug. For change in lifestyle behaviour the system may give the user feedback on the efficacy of their behaviour change reinforcing the behavioural change if it has managed to help them work towards their goal.

The system can allow expensive pharmacotherapy to be focused on those patients either pre-assessed to be poor responders to lifestyle modification or for whom lifestyle modification proves inadequate with respect to maintaining target blood glucose levels as well as other markers of disease status/prognosis.

In some embodiments, the system can monitor behavioural patterns which can be used to determine a behavioural “responder” vs. “non-responder”, that is someone who could be more likely to change their lifestyle as opposed to someone who is less likely based on an array of data. The responder may be given greater resources to support behaviour change than the non-responder. In contrast, the non-responder may be monitored closer for progression to drug based therapies, essentially fast tracking them. Furthermore, by assessing whether someone takes a drug or not by the level in their body, they could be classified as a “responder” or “non-responder” to the drug based therapy. Responders would continue on the therapy with non-responders either receiving an increased dosage or withdrawn from treatment. To determine behavioural response, data could be collected on interactions with digital services, voice data recorded during coaching analysed for tone and meaning, sleep data collected by wearable or by data inputted by the user, physical activity, exercise and sedentary behaviour data collected by a wearable device or by data inputted by the user, diet collected by photograph or by data collected by the user, weight by scales connected to the system or by data inputted by the user. Responders are defined by change thresholds.

In some embodiments, the data gathered across multiple patients, i.e. the patient population, can be used to identify other patients having similar or the same clinical factors in order to determine what the most likely effective treatment/lifestyle regime will be for each patient at any given time. For each patient the system 100 will hold data of their clinical factors and the treatment/lifestyle regime that was meant to be followed by that patient along with data on whether and how well the patient conformed with this regime and the effectiveness of the regime on the patient. Using various techniques including machine learning, various aspects for each patient can be predicted in order to select a treatment/lifestyle regime including likely effectiveness of each option or combination of options as well as likely conformance for the same.

The system may not just take into account generally approved treatment algorithms but individual's responses to such algorithms. The system will understand whether the user responds to a drug and/or therapy or not. The system will also understand whether the user is capable of behavioural change. If they are unable to achieve change in lifestyle behaviour the system would provide a greater emphasis on drug based therapies and its conformance. For example, based on learnings from existing users, the system can determine that a user will have greater success with a specific dietary regimen based on the success of previous participants and user data collected by the system. This develops the “people like you had success with programmes like these” algorithm. The algorithm will be based upon user behavioural data combined, where possible, with clinical data collected either by an IVD or from clinical records.

In the same way that the digital medical record databases 130a, 130b, 130c, 130d are connected to the server 110, the user devices 140, 150, 160, 170 can also communicate with the server 110 through the distributed network 120. Through this connection all user generated inputs gathered by any combination of devices 140, 150, 160 and 170 can be sent to the server 110.

Upon receiving the new user inputs, the server 110 processes the data by comparing it to existing data held on the digital medical record databases 130a, 130b, 130c, 130d. The digital medical record databases 130a, 130b, 130c, 130d may include target ranges or parameters set by the patient's clinician/doctor/medical advisor which are used as benchmarks for the system 100 to determine the effectiveness of a treatment for a patient or the patient conformance to a treatment. The processing of data at the server 110 determines the type of treatments the system 100 will recommend, which have been previously discussed.

At present, clinical care teams assume that patient users take their drugs although we know that this is not the case and poor conformance is prevalent in diabetes diagnosed patients. This makes it difficult to determine whether the drug is not working effectively or whether they have just not consumed the drug. The system can provide awareness of both conformance to drug use and also behavioural changes and thus overcome current issues. By doing so the system is able to provide insight into the real-world efficacy of the therapy. For example, the system can provide immediate feedback on conformance with behavioural and/or drug therapies towards the specified goal by linking a physiological parameter, i.e. glucose, t behaviour or drug use. The interval based approach may provide more information about the overall treatment trajectory. For example, being aware that lifestyle behaviours remain stable and drugs were taken whilst physiological control remains poor would mean that the user would receive a message to either change their lifestyle behaviours further or work with the care team on drug dosing.

In addition to processing the user data, the server 110 can also update the digital medical record databases 130a, 130b, 130c, 130d with new user information and in doing so enables a clinician/doctor/medical advisor to monitor the patient's response to a treatment in real-time. Adjustments to the treatment plan and/or targets can be made accordingly.

In alternative embodiments, other user variables can be consider including weight, height, whether a user smokes, resting heart rate, blood glucose data, age, medical conditions, medical diagnoses, among others.

In some embodiments, data can be collected from one or more devices per user or one device per user or on differing numbers of devices per user.

In some embodiments, the data that is collected from the one or more devices per user can include heart rate, blood glucose level, number of steps per day, intensity of activity, dietary information, among other data.

One or more effectiveness data can be predicted for each user, depending on the application to which the embodiment is being used. For example, when monitoring an exercise regime, user variables such as weight and body dimensions can be used to determine predictions for the expected result of a diet and/or exercise regime. Thus predicted data can be generated based on the expected results (i.e. effectiveness data) and the user devices can measure actual data for a user such as weight, exercise (number of steps per day, number of visits to a gym, heart rate, intensity of exercise), diet (estimated calories consumed), body measurements, among other data (depending mainly on the user devices available). The effectiveness data can then be compared to the actual data to determine an effectiveness of the diet and/or exercise regime. Thus, the diet and/or exercise regime can be adapted or changed on the basis of this effectiveness data.

Any system feature as described herein may also be provided as a method feature, and vice versa. As used herein, means plus function features may be expressed alternatively in terms of their corresponding structure.

Any feature in one aspect may be applied to other aspects, in any appropriate combination. Method aspects may be applied to system aspects, and vice versa. Furthermore, any, some and/or all features in one aspect can be applied to any, some and/or all features in any other aspect, in any appropriate combination.

It should also be appreciated that combinations of the various features described and defined in any aspects of the invention can be implemented and/or supplied and/or used independently.

1. A method of monitoring one or more users comprising the steps of:
receiving user variable data for each of the one or more users;
receiving data from one or more user devices for each of the one or more users;
predicting effectiveness data for each of the one or more users based on the user variable data for each of the one or more users;
comparing received data with predicted effectiveness data for each of the one or more users; and
determining an effectiveness value for each of the one or more users based on the comparison of the received data with the predicted effectiveness data.

2. The method according to claim 1, wherein the one or more user devices include any of: in-vitro diagnostics devices; smartphones; tablet computers; personal computers.

3. The method according to claim 1, further comprising a step of determining a state for each of the one or more users, wherein the state correlates to a need for a treatment.

4. The method according to claim 1, wherein the treatment comprises a lifestyle regime and/or a drug(s) regime.

5. The method according to claim 1, wherein the effectiveness value determines a modification of the lifestyle regime and/or the drug(s) regime.

6. The method according to claim 1, wherein the data from the one or more user devices is any of: input manually by the user; and/or collected as sensor data from the one or more user devices.

7. The method according to claim 1, wherein the predicted effectiveness data corresponds to responses to a drug(s) regime and/or a goal-orientated response to a lifestyle regime.

8. The method according to claim 1, further comprising the step of determining one or more negative responses to the treatment.

9. The method according to claim 1, further comprising a step of categorising the one or more users into one or more user categories based on the responses to the drug(s) regime and/or the goal orientated response to a lifestyle regime.

10. The method according to claim 9, wherein one or more commonalities is determined for each of the one or more user categories, further wherein the one or more commonalities determines a modification of the lifestyle regime and/or the drug(s) regime.

11. The method according to claim 1, wherein the data comprises biomarker data.

12. The method according to claim 1, wherein the treatment comprises the use of any of: SGLT2 inhibitors; DPP-4 inhibitors; and/or GLP-1 receptor agonists.

13. A method of monitoring one or more users in respect to conformance with a treatment, comprising the steps of:
receiving user variable data for each of the one or more users;
receiving data from one or more user devices for each of the one or more users;
predicting effectiveness data for each of the one or more users based on the user variable data for each of the one or more users;
comparing received data with predicted effectiveness data;
determining conformance with the treatment for each of the one or more users based on the comparison of the received data with the predicted effectiveness data based on the comparison.

14. The method according to claim 13, wherein the treatment comprises a lifestyle regime and/or a drug(s) regime.

15. The method according to claim 13, wherein the received data is compared to any one of predicted effectiveness data and predetermined conformance data.

16. The method according to claim 13, wherein the predicted effectiveness data and the predetermined conformance data corresponds to a response to a drug(s) regime and/or a goal-orientated response to a lifestyle regime.

17. The method according to any of claim 13, wherein the step of determining conformance of the treatment based for each of the one or more users based on the comparison of the received data with the predicted effectiveness data further determines a modification of the lifestyle regime and/or the drug(s) regime.

18. The method according to any of claim 13, wherein the treatment further comprises the use of any of: SGLT2 inhibitors; DPP-4 inhibitors; GLP-1 receptor agonists.

19. A method of determining a treatment for one or more users comprising the steps of:
receiving user variable data for each of the one or more users;
receiving data from one or more user devices for each of the one or more users;
predicting effectiveness data for each of the one or more users based on the user variable data for each of the one or more users;
comparing the received data with predicted effectiveness data;
determining an effectiveness value for each of the one or more users based on the comparison of the received data with the predicted effectiveness data based on the comparison; and
determining the treatment for the one or more users in dependence upon the effectiveness value.

20. The method according to claim 19, wherein the treatment comprises a lifestyle regime and/or a drug(s) regime.

21-45. (canceled)
